(19) United States
(12) Patent Application Publication
(10) Pub. No.: US 2022/0304562 A1
(43) Pub. Date: Sep. 29, 2022

(54) ENDOSCOPE

(71) Applicant: Pro-Sight Medical Technology CORP.,LTD., Miaoli County (TW)

(72) Inventors: Sheng Wen Huang, Miaoli County (TW); Chen-Chung Hsu, Hsinchu County (TW); Ya-Hsuan Lee, Hsinchu City (TW)

(73) Assignee: Pro-Sight Medical Technology CORP.,LTD., Miaoli County (TW)

(21) Appl. No.: 17/699,198

(22) Filed: Mar. 21, 2022

(30) Foreign Application Priority Data
Mar. 23, 2021 (TW) 110110403

(51) Int. Cl. A61B 1/04; A61B 1/05; A61B 1/00; A61B 1/045; A61B 1/06

(52) U.S. Cl. CPC A61B 1/041; A61B 1/05; A61B 1/00114; A61B 1/045; A61B 1/00018; A61B 1/0676

(57) ABSTRACT

An endoscope includes a control module, a lens module, a cover, and an encapsulation. The lens module is electrically connected to the control module. The cover has a transparent area, and the cover covers the lens module in a sealing manner. The encapsulation encapsulates the cover and the control module, and exposes the transparent area, such that the encapsulation serves as a shell of the endoscope.

### CROSS-REFERENCE TO RELATED APPLICATION

This application claims the priority benefit of Taiwanese application serial no. 110110403, filed on Mar. 23, 2021. The entirety of the above-mentioned patent application is hereby incorporated by reference herein and made a part of this specification.

### BACKGROUND

### Technical Field

The disclosure relates to an endoscope, and in particular to an endoscope that is easy to swallow and has better heat dissipation and image quality.

### Description of Related Art

Currently, in the capsule form of endoscopy, the main electronic components are located in one of the capsule shells, and the other transparent capsule shell is fixed to the capsule shell containing the main electronic components to form a complete capsule. The transparent capsule shell covers the lens to enable the lens to capture the external image. The two capsule shells are mainly bonded to each other by dispensing or ultrasonic welding.

However, since the two capsule shells are filled with air after bonding, the capsule endoscope will float on the upper side of the oral cavity, further irritating the throat and causing difficulty in swallowing. In addition, when the capsule endoscope is in operation, the temperature of the electronic components rises and the moisture in the air inside the capsule endoscope condenses on the inner wall of the transparent capsule shell, causing blurred images, which further affects the accuracy of medical diagnosis. Furthermore, the air inside the capsule endoscope also makes the heat generated by the electronic components not easy to dissipate, easily causing the image sensor to be heated, which affects the image quality.

### SUMMARY

The disclosure provides an endoscope that is easy to swallow and has better heat dissipation and image quality.

An endoscope of the disclosure includes a control module, a lens module, a cover, and an encapsulation. The lens module is electrically connected to the control module. The cover has a transparent area, and the cover covers the lens module in a sealing manner. The encapsulation encapsulates the cover and the control module, and exposes the transparent area, such that the encapsulation serves as a shell of the endoscope.

In one embodiment of the disclosure, the cover includes an opening end, the control module includes a control circuit board and a control chip electrically connected to the control circuit board, and the opening end of the cover is sealed to the control circuit board.

In one embodiment of the disclosure, the endoscope further includes a magnet disposed next to the cover and the lens module, and the encapsulation encapsulates the magnet.

In one embodiment of the disclosure, the endoscope further includes a battery disposed next to the cover and the lens module, and the encapsulation encapsulates the battery.

In one embodiment of the disclosure, the endoscope further includes a light source module electrically connected to the control module and adjacent to the lens module. The cover covers the light source module.

In one embodiment of the disclosure, the endoscope further includes a wire electrically connected to the control module and away from the lens module. The encapsulation encapsulates a part of the wire.

In one embodiment of the disclosure, the outside of the encapsulation has no additional shell.

In one embodiment of the disclosure, an outer surface of the transparent area of the cover is a flat surface, the encapsulation has a cylindrical outer wall surface and a dome surface away from the flat surface, and the cylindrical outer wall surface and the dome surface are smooth and joint-free surfaces respectively.

In one embodiment of the disclosure, a material of the encapsulation comprises liquid silicone.

In one embodiment of the disclosure, the endoscope is a capsule endoscope.

Based on the above, the endoscope of the disclosure uses the encapsulation to encapsulate the cover and the control module, and expose the transparent area of the cover, such that the encapsulation serves as the shell of the endoscope. Since the elements of the endoscope are encapsulated by the encapsulation, the air inside the endoscope exists only between the cover and the lens module, and the air volume is very low and the buoyancy in the liquid is quite small. Therefore, the endoscope of this disclosure does not float on the upper side when passing through the user's oral cavity, thus achieving an effect of easy swallowing. In addition, because the heat transfer method of solids is mainly heat conduction, while the gas can only transfer heat by heat convection or heat radiation, and the speed of heat conduction is greater than the speed of heat convection or heat radiation, solids have better thermal conductivity compared to gases. The high volume of air inside the conventional endoscope makes it difficult for the internal heat to be transferred, resulting in poor heat dissipation, and the moisture in the air of the conventional endoscope tends to attach to the transparent cover at high temperatures and affects the image quality. The internal air volume of the endoscope of the disclosure is low, and the internal heat can be transferred through the encapsulation by heat conduction, which has better heat dissipation, and because the internal air volume is very low, moisture is not easily generated, and the image quality can be kept stable.

To make the aforementioned more comprehensible, several accompanied with drawings are described in detail as follows.

### BRIEF DESCRIPTION OF THE DRAWING

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 to FIG. 4B are schematic views of an encapsulating process of an endoscope according to an embodiment of the disclosure.

FIG. 5 is a three-dimensional schematic view of the endoscope of FIG. 4A.

FIG. 6 to FIG. 7 are schematic views of an encapsulating process of an endoscope according to another embodiment of the disclosure.

### DESCRIPTION OF THE EMBODIMENTS

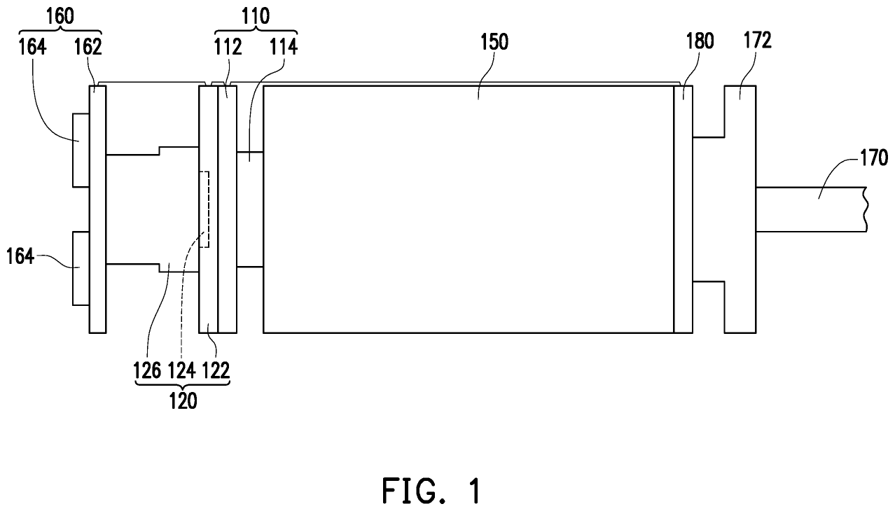

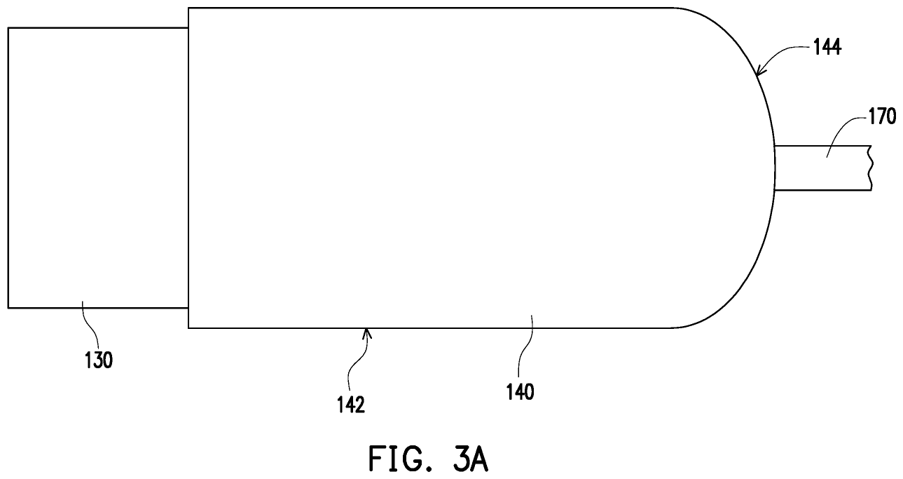

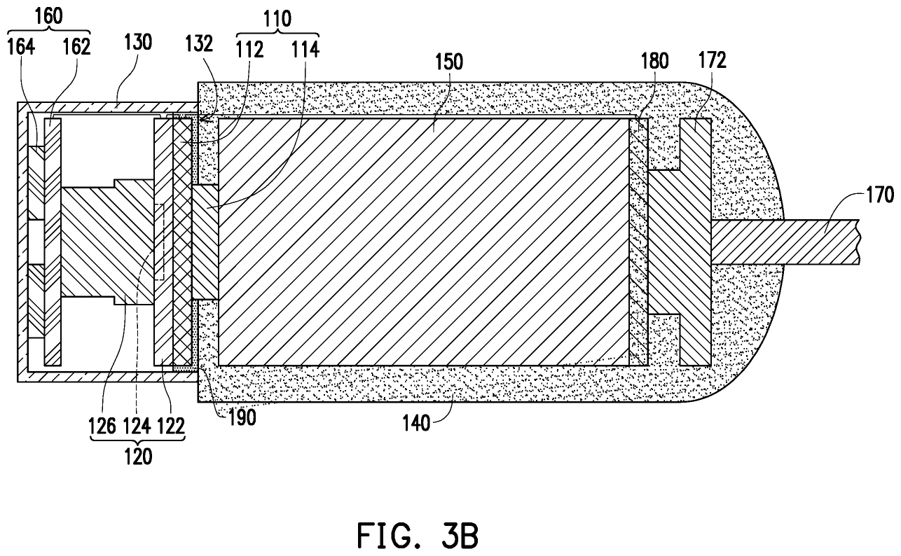

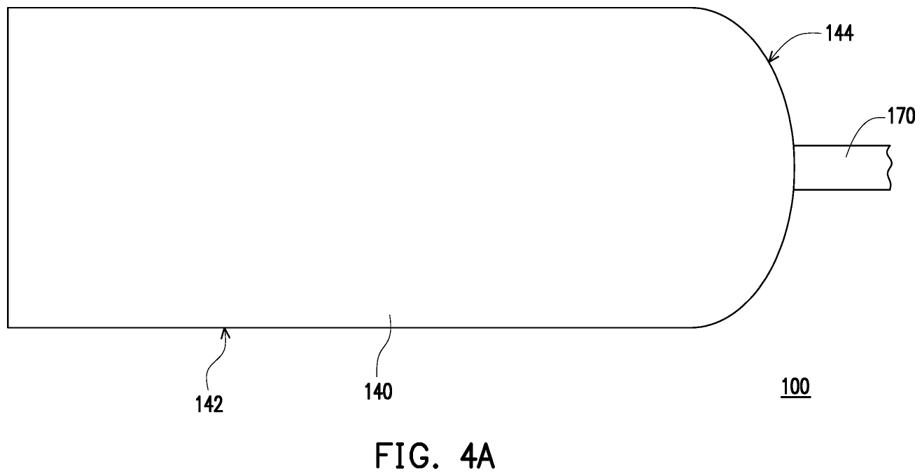

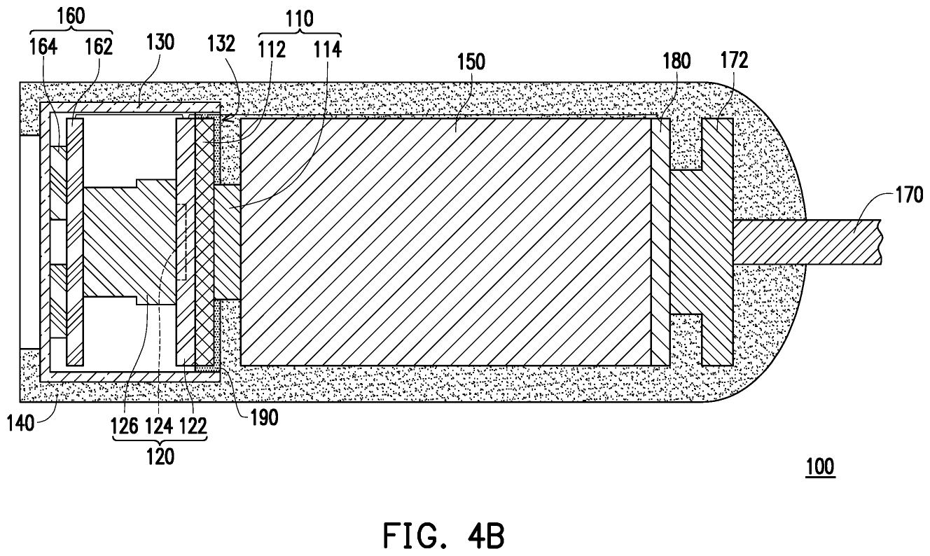

FIG. 1 to FIG. 4B are schematic views of an encapsulating process of an endoscope according to an embodiment of the disclosure. FIG. 3B and FIG. 4B are cross-sectional views of FIG. 3A and FIG. 4A, respectively. It should be noted that, in this embodiment, an endoscope 100 is a capsule endoscope for example, but the type of the endoscope 100 is not limited thereto. In other embodiments, the endoscope 100 may also be a handheld endoscope. That is, the front end of the handheld endoscope may also be encapsulated in the manner of this embodiment and have a shell formed by an encapsulation 140 (marked in FIG. 4B).

Referring to FIG. 1 first, FIG. 1 is shows an internal structure of an endoscope when not yet encapsulated. In this embodiment, the internal structure of the endoscope 100 (FIG. 4B) may optionally include a control module 110, a lens module 120, a magnet 150, a light source module 160, and a wire 170. The lens module 120 is electrically connected to the control module 110. Specifically, the control module 110 includes a control circuit board 112 and a control chip 114 disposed and electrically connected to the control circuit board 112.

The lens module 120 includes a lens body 126 and a circuit board 122, and the circuit board 122 includes a photosensitive element 124. The lens body 126 corresponds to the photosensitive element 124 of the circuit board 122. The circuit board of the lens module 120 is electrically connected to the control circuit board 112 to transfer information sensed by the photosensitive element 124 to the control chip 114.

The light source module 160 is disposed on a front side of the lens module 120 and adjacent to the lens module 120 so as to provide light to the lens module 120. The light source module 160 includes a light source 164 and a light source circuit board 162. The light source circuit board 162 is electrically connected to the control circuit board 112.

The wire 170 is electrically connected to the control module 110 and away from the lens module 120. Specifically, the wire 170 is connected to an adapter board 180 through an electrical connector 172, the adapter board 180 is electrically connected to the control circuit board 112, and signals of the control circuit board 112 may be transmitted to the outside world through the adapter board 180 and the wire 170. Alternatively, commands from the outside world may be transmitted to the control circuit board 112 through the wire 170 and the adapter board 180 to control the endoscope 100.

In other embodiments, the endoscope 100 may also not have the wire 170, but may instead transmits information to or receives information from the outside world by means of wireless communication. Alternatively, in one embodiment, the information captured by the endoscope 100 during operation may be stored in an internal storage medium (e.g., a memory), and after the operation is completed, an operator then removes the information stored in the internal. Of course, the form of the endoscope 100 is not limited thereto.

The magnet 150 may be used to rotate the angle and direction of the endoscope 100. For example, when the endoscope 100 is operated, the operator may place another magnetic element at a specific position outside the human body, so that the magnet 150 in the endoscope 100 is turned to a specific angle or direction to enable the lens module 120 to capture the desired image. Of course, in other embodiments, the endoscope 100 may be controlled in other ways, and the magnet 150 can be omitted. In addition, the magnet 150 may also be used to increase the weight to increase the overall density.

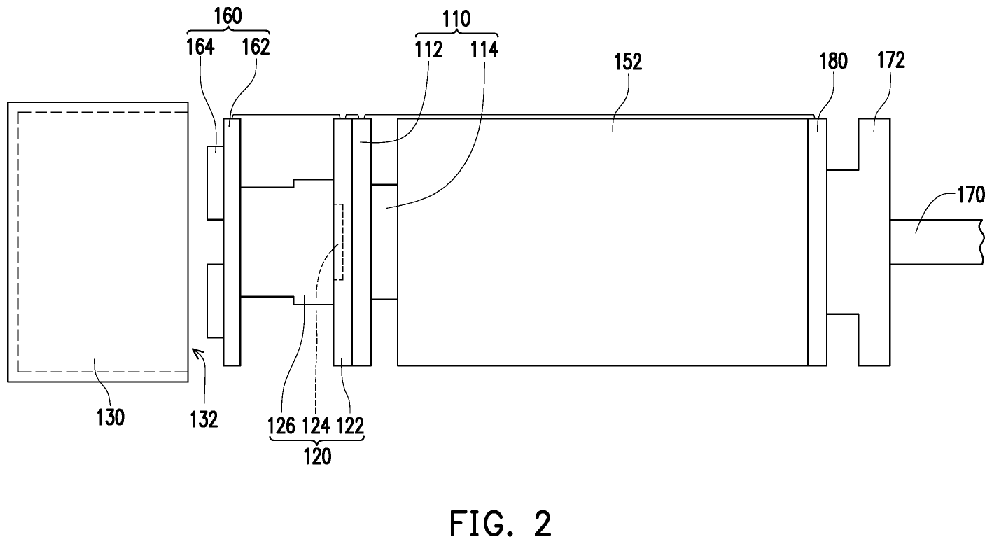

Referring to FIG. 2, a cover 130 is used to cover the lens module 120 prior to encapsulating. Specifically, the cover 130 may be put in from the left side of FIG. 2, and the cover 130 may cover the light source module 160, the lens module 120, and the control module 110. In addition, the cover 130 includes an opening end 132. The opening end 132 of the cover 130 is sealed to the control circuit board 112 through an adhesive 190 (FIG. 3B) to cover the light source module 160, the lens module 120, and the control module 110 in a sealing manner. Of course, in other embodiments, the opening end 132 of the cover 130 may also be sealed to the magnet 150 or other elements through the adhesive 190 or other means, not to be limited thereby.

Next, referring to FIG. 3A and FIG. 3B, internal elements of the endoscope 100 are encapsulated. In this embodiment, the middle part and the right side part of FIG. 3A and FIG. 3B may be encapsulated by clamping the cover 130, so that the encapsulation 140 covers the magnet 150, the adapter board 180, the electrical connector 172, and a part of the wire 170.

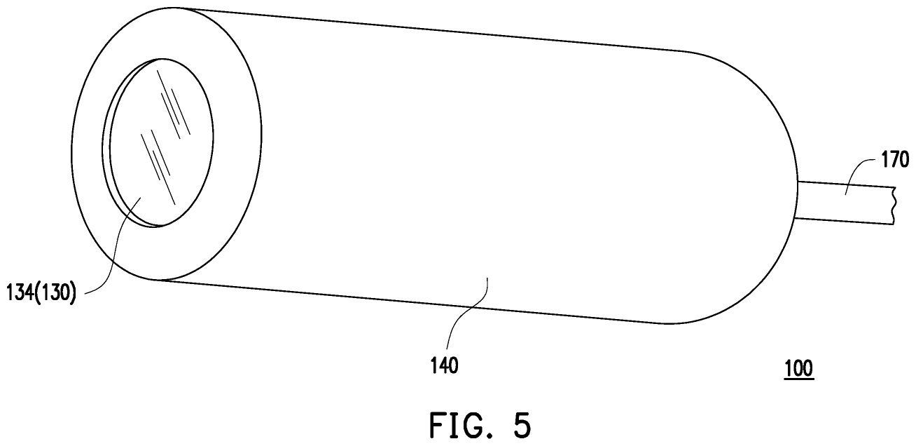

Next, referring to FIG. 4A and FIG. 4B, another part of the cover 130 is encapsulated, so that the encapsulation 140 covers the cover 130. FIG. 5 is a three-dimensional schematic view of the endoscope of FIG. 4A. Referring to FIG. 5, the cover 130 has a transparent area 134. The encapsulation 140 exposes the transparent area 134 of the cover 130 to avoid obscuring the view of the lens module 120, and the lens module 120 is able to capture images outside the transparent area 134 of the cover 130.

As is clear from FIG. 4A to FIG. 5, in this embodiment, the encapsulation 140 serves as the shell of the endoscope 100. That is, the outside of the encapsulation 140 has no additional shell. Compared to the conventional endoscope with a plastic shell (e.g., PC) and air between the plastic shell and the internal elements, the shell of the endoscope 100 in this embodiment is the encapsulation 140. Since the encapsulation 140 encapsulates the internal elements of the endoscope 100 (the magnet 150, the adapter plate 180, the electrical connector 172, and the cover 130), an outer surface of the encapsulation 140 is filled with only the material of the encapsulation 140 between the magnet 150, the adapter board 180, the electrical connector 172, and the cover 130, and no air exists between the encapsulation 140 and the magnet 150, the adapter board 180, the electrical connector 172, and the cover 130.

In addition, in this embodiment, since the cover 130 is sealed to the control circuit board 112, the air inside the endoscope 100 exists only between the cover 130 and the control circuit board 112, and the air volume is very low and the buoyancy in the liquid is quite small. In addition, the exterior of the cover 130 is encapsulated with the encapsulation 140, which increases the overall density. Therefore, when passing through the user's oral cavity, the endoscope 100 does not float on the upper side due to its low buoyancy and high density, thus achieving an effect of easy swallowing.

Furthermore, because the heat transfer method of solids is mainly heat conduction, while the gas can only transfer heat by heat convection or heat radiation, and the speed of heat conduction is greater than the speed of heat convection or heat radiation, solids have better thermal conductivity compared to gases. The high volume of air inside the conventional endoscope makes it difficult for the internal heat to be transferred, resulting in poor heat dissipation, and the moisture in the air of the conventional endoscope tends to attach to the transparent cover at high temperatures and affects the image quality. The internal air volume of the endoscope 100 of the disclosure is low, and the internal heat can be transferred through the encapsulation 140 by heat conduction, which has better heat dissipation, and because the internal air volume is very low, moisture is not easily generated, and the image quality can be kept stable.

In addition, in this embodiment, a material of the encapsulation 140 includes liquid silicone. Liquid silicone is liquid before the seal is formed, which has better flowability and may completely cover the internal elements of the endoscope 100 to reduce the chance of incomplete encapsulating and achieve an effect of full encapsulating. Moreover, the liquid silicone has good biocompatibility and is resistant to acids and alkalis, effectively avoiding the chance of being corroded by stomach acids. Of course, in other embodiments, the encapsulation 140 can also be other materials, not to be limited thereby.

As can be seen from FIG. 4A, in this embodiment, the encapsulation 140 has a cylindrical outer wall surface 142 and a dome surface 144 away from a flat area. In this embodiment, the cylindrical outer wall surface 142 and the dome surface 144 are smooth and joint-free surfaces respectively. Furthermore, as can be seen in FIG. 5, an outer surface of the transparent area 134 of the cover 130 is a flat surface, and the overall shape of the endoscope 100 is flat at one end and hemispherical at the other end. Of course, in other embodiments, if the encapsulation 140 is transparent, the cover 130 may also be completely covered, so that the appearance of the endoscope 100 is in the shape of a capsule with hemispheres at both ends.

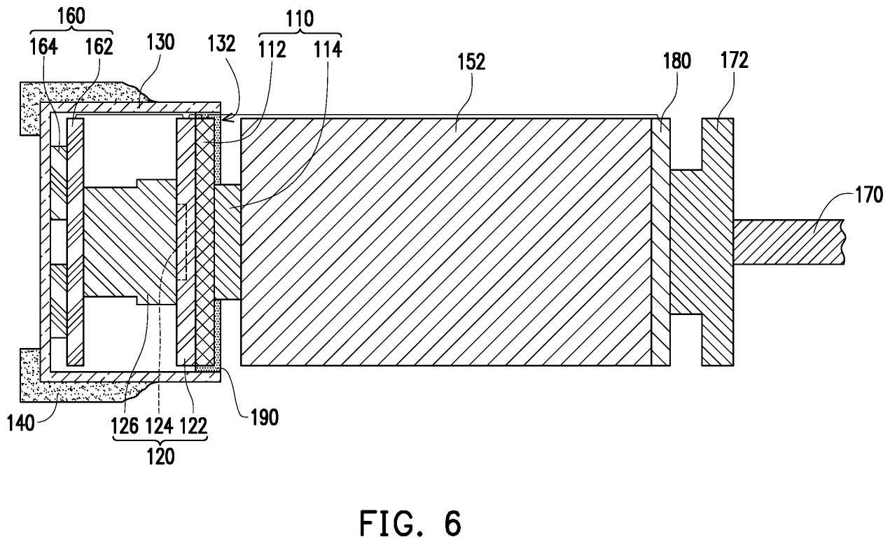

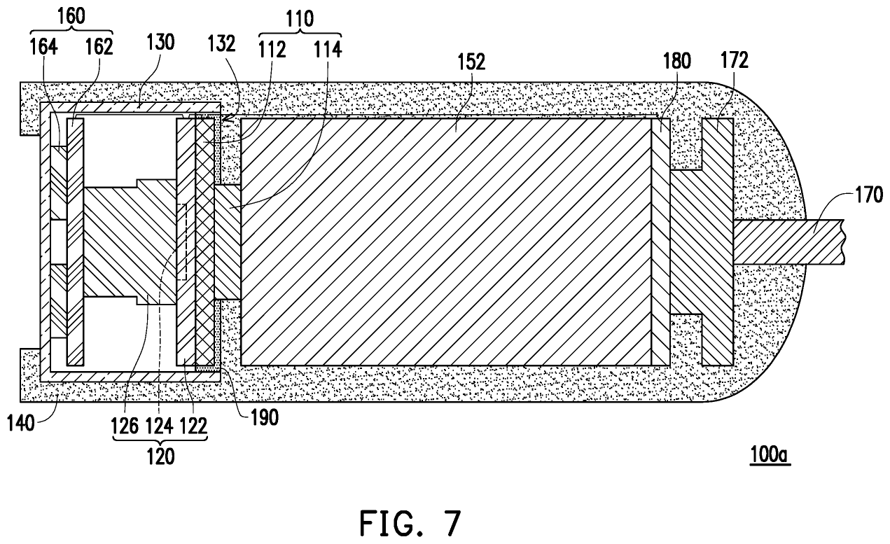

FIG. 6 to FIG. 7 are schematic views of an encapsulating process of an endoscope according to another embodiment of the disclosure. Referring to FIG. 6 first, the main difference between FIG. 6 and FIG. 3B is that the magnet 150 of FIG. 3B may be replaced with a battery 152 of the endoscope 100 according to this embodiment. Such a design allows the battery 152 in the endoscope 100 to supply power to the internal elements on its own. Similarly, the battery 152 also has the effect of increasing the weight, so that the endoscope 100 may have a certain weight and is not easily floating on the liquid surface.

In addition, in this embodiment, when the encapsulation procedure is carried out, the cover 130 may also be encapsulated first as shown in FIG. 6. Next, as shown in FIG. 7, the middle part and the right part of the internal elements of an endoscope 100a are encapsulated, and the encapsulation 140 is encapsulated to the battery 152, the adapter board 180, the electrical connector 172, and a part of the wire 170, and the endoscope 100a is manufactured. Of course, the order and number of encapsulation are not limited, and in other embodiments, the entire structure may also be encapsulated at the same time.

To sum up, the endoscope of the disclosure uses the encapsulation to encapsulate the cover and the control module, and expose the transparent area of the cover, such that the encapsulation serves as the shell of the endoscope. Since the components of the endoscope are encapsulated by the encapsulation, the air inside the endoscope exists only between the cover and the lens module, and the air volume is very low and the buoyancy in the liquid is quite small. Therefore, the endoscope of this disclosure does not float on the upper side when passing through the user's oral cavity, thus achieving an effect of easy swallowing. In addition, because the heat transfer method of solids is mainly heat conduction, while the gas can only transfer heat by heat convection or heat radiation, and the speed of heat conduction is greater than the speed of heat convection or heat radiation, solids have better thermal conductivity compared to gases. The high volume of air inside the conventional endoscope makes it difficult for the internal heat to be transferred, resulting in poor heat dissipation, and the moisture in the air of the conventional endoscope tends to attach to the transparent cover at high temperatures and affects the image quality. The internal air volume of the disclosure is low, and the internal heat can be transferred through the encapsulation by heat conduction, which has better heat dissipation, and because the internal air volume is very low, it is not easy to generate moisture, and the image quality can be kept stable.

It will be apparent to those skilled in the art that various modifications and variations can be made to the disclosed without departing from the scope or spirit of the disclosure. In view of the foregoing, it is intended that the disclosure covers modifications and variations provided that they fall within the scope of the following claims and their equivalents.

What is claimed is:

1. An endoscope comprising:
a control module;
a lens module electrically connected to the control module;
a cover having a transparent area, wherein the cover covers the lens module in a sealing manner; and
an encapsulation encapsulating the cover and the control module, and exposing the transparent area, such that the encapsulation serves as a shell of the endoscope.

2. The endoscope according to claim 1, wherein the cover comprises an opening end, the control module comprises a control circuit board and a control chip electrically connected to the control circuit board, and the opening end of the cover is sealed to the control circuit board.

3. The endoscope according to claim 1 further comprising:
a magnet disposed next to the cover and the lens module, and the encapsulation encapsulating the magnet.

4. The endoscope according to claim 1 further comprising:
a battery disposed next to the cover and the lens module, and the encapsulation encapsulating the battery.

5. The endoscope according to claim 1 further comprising:
a light source module electrically connected to the control module and adjacent to the lens module, wherein the cover covers the light source module.

6. The endoscope according to claim 1 further comprising:
a wire electrically connected to the control module and away from the lens module, wherein the encapsulation encapsulates a part of the wire.

7. The endoscope according to claim 1, wherein the outside of the encapsulation has no additional shell.

8. The endoscope according to claim 1, wherein an outer surface of the transparent area of the cover is a flat surface, the encapsulation has a cylindrical outer wall surface and a dome surface away from the flat surface, and the cylindrical outer wall surface and the dome surface are smooth and joint-free surfaces respectively.

9. The endoscope according to claim 1, wherein a material of the encapsulation comprises liquid silicone.

10. The endoscope according to claim 1, wherein the endoscope is a capsule endoscope.
